(19) United States
(12) Patent Application Publication
(10) Pub. No.: US 2011/0030697 A1
(43) Pub. Date: Feb. 10, 2011

(54) Manufacture, testing, assembly, packaging, sale and method of use of condom with integrated applicator

(76) Inventor: Robert Beau Thompson, Miami, FL (US)

Correspondence Address: Robert J. Van Der Wall;Grand Bay Plaza, Suite 701, 2665 South Bayshore Drive, Coconut Grove, FL, 33133, US

(21) Appl. No.: 12/462,613

(22) Filed: Aug. 7, 2009

(51) Int. Cl. A61F 6/04; B26D 7/27; B65B 51/10; B65B 5/08

(52) U.S. Cl. 128/844; 53/396; 53/463; 53/473

(57) ABSTRACT

The condom of the present invention is manufactured, tested, assembled and packaged using the apparatus and method of the present invention with an integrated condom applicator assembly. That is formed of two strips or tabs of flexible material rolled into the rolled condom such that tab ends protrude from the roll. The tab ends are provided with a touch distinctive tactile or textured surface on one side thereof so that a user can orient the rolled condom in the proper direction in the dark. A method of condom assembly is included in which the tabs are manufactured and rolled into the rolled condom to form the tab applicator assemblies using a custom designed machine. The inventive process next contemplates a packaging machine, which wraps the combined condom and applicator assembly in a sealed package. The marketing aspect of the invention is structured using manufacturer's representatives. Finally, the process includes instructing the consuming public on the method of use of the condom.

### BACKGROUND OF THE INVENTION

1. Field of the Invention

The present invention relates generally to a process for the manufacture, testing, assembly, packaging, sale and instruction of the public on the method of use of a condom with integrated applicator. The condom with integrated applicator is as described in U.S. Pat. No. 5,758,659 (“the '659 patent”). The condom of the present invention is unique because of the integrated condom applicator, which assures proper orientation of the condom using the sense of touch and facilitates application of the condom to a penis in the dark.

The above described '659 patent describes the structure of the condom of the present invention, but it does not describe an economical and commercially viable way to manufacture, test and integrate the tab applicator assembly with a condom. The problem has been the absence of a commercially viable method and apparatus both for economically manufacturing tab applicator assemblies and for economically integrating them with existing condoms after testing the condoms. Part of the present invention addresses those problems with a custom designed machine.

The condom itself is of generally conventional design, having a condom tubular side wall, a closed end with an end wall and an open end with a side wall rim. The condom is provided with the tubular side wall rolled outwardly and over itself from the side wall rim toward the end wall to form a side wall roll.

The inventive condom applicator assembly is formed of two strips of flexible material, rolled with the tubular side wall into the side wall roll such that tab end segments protrude from the roll. The tab end segments are preferably provided with a tactile textured surface on one side thereof so that a user can orient the side wall roll in the proper direction in the dark. A method of condom assembly is described in which the strips are manufactured and rolled into the side wall roll to form the tab applicator assemblies and to properly integrate them with existing condoms using a custom designed machine.

The inventive process next contemplates a packaging machine, which wraps the combined condom and applicator assembly in a sealed package. The packages are combined into boxes of three packs or twelve packs for shipment and delivery purposes. The marketing aspect of the invention is structured using manufacturer's representatives.

Finally, the process includes instructing the consumer on the method of use of the condom. To use the condom, the package is opened, and the user places the condom end wall against the distal end of the penis, so that the side wall roll is directed away from the user. This is accomplished in the dark by the user's sense of touch of the optional tactile textured surfaces when the user grasps the two tab end segments. The user then pulls the two tab end segments laterally outward from the sides of the penis. This single action causes the side wall roll to rapidly unroll around and longitudinally along the penis.

2. Description of the Prior Art

There have long been various types of condoms and condom packages. Condoms have included lubricated and unlubricated versions and have been made of various materials, such as latex. Condom packages have included sealed envelopes, wrappers and flat metal containers. A problem with these prior condoms and condom packages has been that they do not assist the user in condom application, and application is difficult or impossible in the dark because the user cannot see which way to orient the condom. Further, as pointed out above, the prior art has been lacking in a commercially viable way to economically manufacture tab applicator assemblies and to economically integrate them with existing condoms.

It is thus a principal object of the present invention to provide a condom including an integral applicator assembly which can be mass produced economically.

It is another object of the present invention to provide such a condom in which the applicator assembly does not significantly increase the size of the condom package.

It is a further object of the invention to provide a means for orienting the condom so that the applicator can be reliably and easily operated by touch in the dark.

It is an additional object of the present invention to provide a product that combines a condom with the applicator assembly and sealed packaging inexpensively.

It is another object of the present invention to provide a method and apparatus for manufacturing condoms with tab applicator assemblies that is both efficient and cost effective.

It is a further object of the present invention to make available to condom users condoms that employ the tab applicator assembly. The tab applicator assembly provides an easier method of applying a condom. If a condom is easier to apply, then it follows that it is more likely to be used. Condoms use substantially decreases the chance of contracting a sexually transmitted disease and is a proven method of birth control.

It is another object of the present invention is to test each condom prior to adding a tab applicator assembly to it and to reject any condom found to be defective. The use of defective condoms can result in ineffective protection against disease. Such use can also result in unplanned pregnancies.

A further object of the present invention is to provide a method and apparatus for rolling up other types of items as well as condoms, items such as balloons and rubber gloves.

An additional object of this invention is to provide tabs that can be rolled up inside of condoms or other types of items, such as balloons or rubber gloves, to aid in unrolling the item. By taking hold of one or more tabs and pulling, a rolled-up item of the type mentioned above can be more easily unrolled.

Another object of the invention is to provide a method and apparatus for cutting tabs from suitable materials other than latex or plastic, such as paper or felt. These tabs can then be rolled up inside of condoms or other types of items, such as balloons or rubber gloves.

Another object of the invention is to provide tabs of material to be rolled up inside of condoms or other types of items, such as balloons or rubber gloves. Such tabs can display instructions for use, advertisements, jokes, cartoons, horoscopes, and other such printable matter.

Another object of the invention is to provide novelty tabs to be rolled up inside of condoms or other types of items, such as balloons or rubber gloves. For instance, such tabs may have unusual characteristics, such as a fluorescent color, an uncommon texture, a pleasant scent, or the like.

It is another object of the invention to combine the sealed packages into boxes of three packs or twelve packs for shipment and delivery purposes with the marketing aspect of the invention structured using manufacturer's representatives hired by the owner of this invention and the foregoing equipment.

Finally, the process includes instructing the consuming public on the method of use of the condom as described in detail hereinafter.

### SUMMARY OF THE INVENTION

The present invention accomplishes the above-stated objectives, as well as others, as may be determined by a fair reading and interpretation of the entire specification.

A condom is provided including a condom tubular side wall having a first side wall end and a second side wall end, the tubular side wall being rolled outwardly and over itself from the second side wall end toward the first side wall end to form a side wall roll; a condom end wall sealingly joined to the tubular side wall at the first side wall end; and at least one strip of flexible material having a gripping tab end segment with a tactile textured surface on one side thereof, at least one strip being rolled within the side wall roll so that the gripping tab end segment of the at least one strip protrudes from the side wall roll such the gripping tab end segment can be easily grasped and the tactile textured surface felt.

The condom preferably includes at least two of the strips of flexible material rolled within the side wall roll, the two strips being positioned on laterally opposing portions of the tubular side wall. The condom preferably is formed of latex condom material and the at least one strip is formed of a plastic material such as high density polyethylene. The at least one strip preferably is of sufficient length to extend at least from the side wall second end to the side wall first end. Tactile textured surfaces are preferably provided at least on the gripping tab end segment thereof.

A method is provided of assembling the condom, including the step of rolling the at least one strip together with the tubular side wall outwardly and over the tubular side wall toward the side wall first end to form a side wall roll, so that the gripping tab end segment and tactile textured surface protrudes from the side wall roll. The tab applicator assembly comprises two narrow strips of plastic material that are rolled-up with the condom. One strip extends outwardly from the left side of the condom and the other strip extends outwardly from the right side. One side of each end of a strip, the gripping tab end segment, is textured so as to allow the user to determine, through sense of touch, if the strip, and thus the condom, is oriented in the proper direction.

In accordance with a new aspect of the present invention, an apparatus manufactures strips of material, known also as tabs, and integrates these tabs with existing condoms that the apparatus has tested for integrity. The apparatus manufactures the tabs by cutting strips from a flexible material, such as latex or plastic. The apparatus then applies a tab to either side of each non-defective condom it processes and rolls up the two tabs with the condom so that the end of each tab remains outside the rolled-up condom. In a preferred embodiment, the end of each tab is textured on one side allowing for the user to determine, through sense of touch, the correct orientation of the condom. In this way, tab applicator assemblies are economically manufactured and integrated with existing condoms.

In accordance with another aspect of the present invention there is provided a packaging apparatus that combines a condom with the applicator assembly in a sealed package (packs). These sealed packs are then further packaged for marketing, shipment and sale in various configurations. Examples are boxes of three packs or twelve packs that are loaded into cartons for shipment and delivery purposes with the marketing aspect of the invention structured using manufacturer's representatives.

In accordance with another aspect of the invention there is provided a technique instructing the purchasing public of a method of applying the condom to a penis, including the steps of grasping the tab end segment, feeling the tactile textured surface thereon, orienting the condom in the proper direction, placing the condom end wall against the distal end of the penis, so that the tubular side wall roll is directed away from the user; and pulling the tab end segment laterally outwardly from the tubular side wall, so that the side wall roll unrolls over and advances longitudinally along the penis. The method preferably includes the additional step of lifting the at least one strip away from the tubular side wall.

### BRIEF DESCRIPTION OF THE DRAWINGS

Various other objects, advantages, and features of the invention will become apparent to those skilled in the art from the following discussion taken in conjunction with the following drawings, in which:

FIG. 1 is a perspective view of the condom and the condom applicator assembly in its rolled configuration as provided to the user after removal from the sealed packaging.

FIG. 2 is a side view of the condom body extended to full length prior to rolling of the tubular side wall, and of two strips to be rolled together with the side wall into a side wall roll as shown in FIG. 1.

FIG. 3 is a cross-sectional side view of the condom body and strips partly assembled, the tubular side wall being partially rolled into a side wall roll and the two strips shown in FIG. 2 being rolled together with and into the tubular side wall.

FIG. 4 is a cross-sectional side view as in FIG. 3, except that the roll is shown partially unrolled in solid lines as the user's hands advance radially outward from the sides of the penis. The fully unrolled tubular side wall is illustrated in broken lines, with the strips released and separated.

FIG. 5 is a bottom view showing the tactile textured surfaces on the tab end segments.

FIG. 6 is a top view of a preferred embodiment of one apparatus for manufacturing tab applicator assemblies and integrating them with existing condoms;

FIG. 7 is a side elevation of a slide and stripper brush assembly, in accordance with the preferred embodiment shown in FIGS. 6 and 15;

FIG. 8 is a side view of a slide and stripper brush assembly, in accordance with the preferred embodiments shown in FIGS. 6 and 15;

FIG. 9 is a front elevation of components used to cut and position plastic tabs on a condom, in accordance with the preferred embodiments shown in FIGS. 6 and 15;

FIG. 10 is a perspective view of a cutting subassembly used to cut and position plastic tabs, in accordance with the preferred embodiments shown in FIGS. 6 and 15;

FIG. 11 is a perspective view of the cut and place subassembly with the slide and stripper subassembly removed, in accordance with the preferred embodiments shown in FIGS. 6 and 15;

FIGS. 12 and 13 are top and perspective views, respectively, of a subassembly used to move condoms through various stages or production, in accordance with the preferred embodiments shown in FIGS. 6 and 15;

FIG. 14 is a perspective view of a pivot bar and tab rotation bar subassembly used to place plastic tabs on a condom, in accordance with the preferred embodiments shown in FIGS. 6 and 15;

FIG. 15 is a side view of a preferred embodiment of a method for utilizing two apparatus for manufacturing tab applicator assemblies and integrating them with existing condoms; and

FIG. 16 is a front view of components used to control and coordinate the operation of the two apparati of FIG. 15.

FIG. 17 is a front view schematic of the foiler packaging machine with the condom sectioned feeder belt removed for clarity.

FIG. 18 is a top view partial schematic showing the condom sectioned feeder belt and pusher/eccentric tong assembly.

FIG. 19 is a front view schematic of the foiler packaging machine control panel.

### DETAILED DESCRIPTION OF THE PREFERRED EMBODIMENTS

As required, detailed embodiments of the present invention are disclosed herein; however, it is to be understood that the disclosed embodiments are merely exemplary of the invention which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

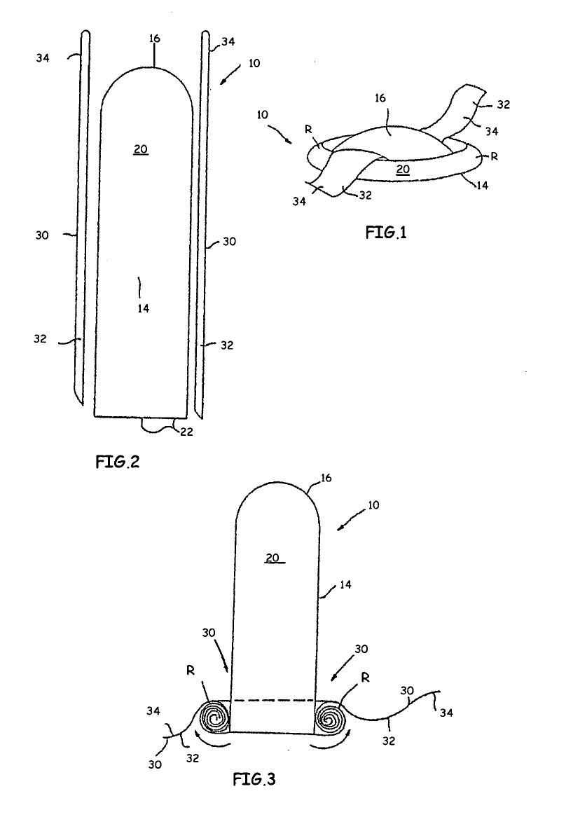

Reference is now made to the drawings, wherein like characteristics and features of the present invention shown in the various figures are designated by the same reference numerals. Referring to FIGS. 1-5, a condom 10 including a condom body 20 and an integrated condom applicator assembly 30 is disclosed. Condom body 20 is of conventional condom design, having a condom tubular side wall 14, a closed first side wall end with a condom end wall 16 and an open second side wall end with a condom tubular side wall rim 22. Condom 10 is provided to the consumer in the conventional product configuration, i.e., with the tubular side wall 14 rolled outwardly and over itself from side wall rim 22 toward end wall 16 to form a side wall roll R, i.e., a rolled condom.

The inventive condom applicator assembly 30 includes at least one, and preferably two, strips 32 of flexible material, such as latex or plastic. Strips 32, also referred to as tabs 32, are separate from the main condom body 20 and are rolled longitudinally with rolled tubular side wall 14 so that gripping tab end segments 34 of strips 32 protrude from side wall roll R. See FIGS. 1-3. Gripping tab end segments 34 have, on one side, tactile textured surfaces 36. See FIG. 5.

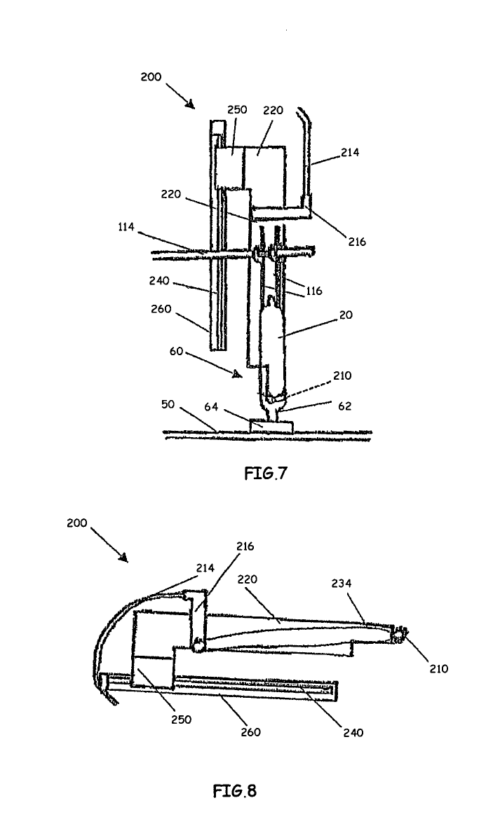

FIGS. 6-16 show a preferred embodiment of a method and apparatus for manufacturing tab applicator assemblies 30 and properly integrating them with existing condom bodies 20.

Referring to FIG. 6, an apparatus 12 is mounted upon a support frame 18. The support frame 18 is canted at an angle of approximately twenty-two degrees. The frame 18 is canted at this angle so as to aid operators in placing condoms 20 on mandrels 62, as explained later. In alternate embodiments, the support frame 18 is oriented at a different angle or oriented horizontally. In some embodiments, the angle of the support frame 18 can be adjusted. A PLC unit 120 controls and coordinates the movements of the various components and mechanisms of the apparatus 12 as the apparatus 12 works to manufacture plastic strips or tabs 32 and integrate the strips or tabs 32 with condom bodies 20. (See FIG. 16 for details of the PLC unit 120.) A track 50 is laid out in a rectangular circuit on the upper surface of the support frame 18. A main drive belt 52 is laid out within the area bounded by the track 50. The path of the belt 52 closely parallels the path of the track 50 so as to form a smaller rectangular circuit within the rectangular circuit formed by the track 50. A main drive sprocket 53 and three idler sprockets 54 are all horizontally aligned with one another. The sprockets 53, 54 are mounted within the path traced by the main drive belt 52, with one sprocket 53, 54 appearing on the inside of each corner of the belt 52. The main drive belt 52 is drawn tautly around each sprocket 53, 54 in a manner that maintains the belt's 52 rectangular shape. In alternate embodiments, it is possible to employ a greater or fewer number of sprockets 53, 54, and a track and belt in different configurations.

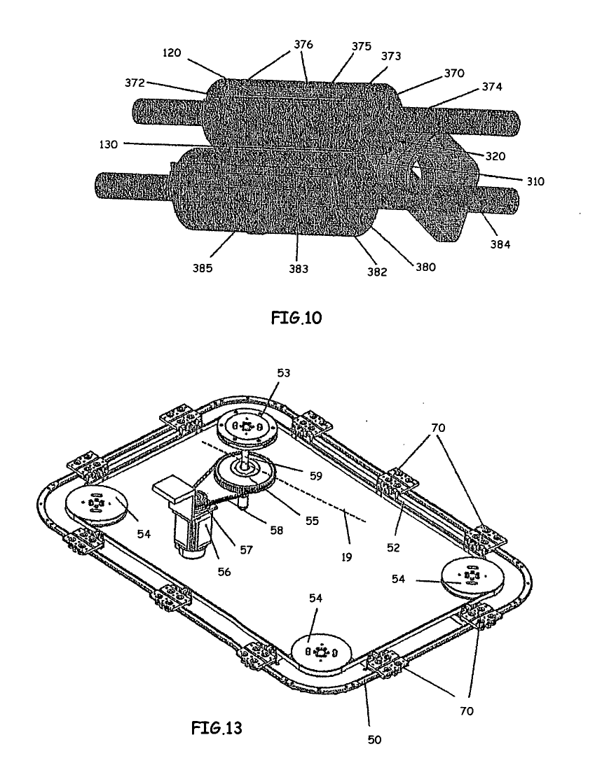

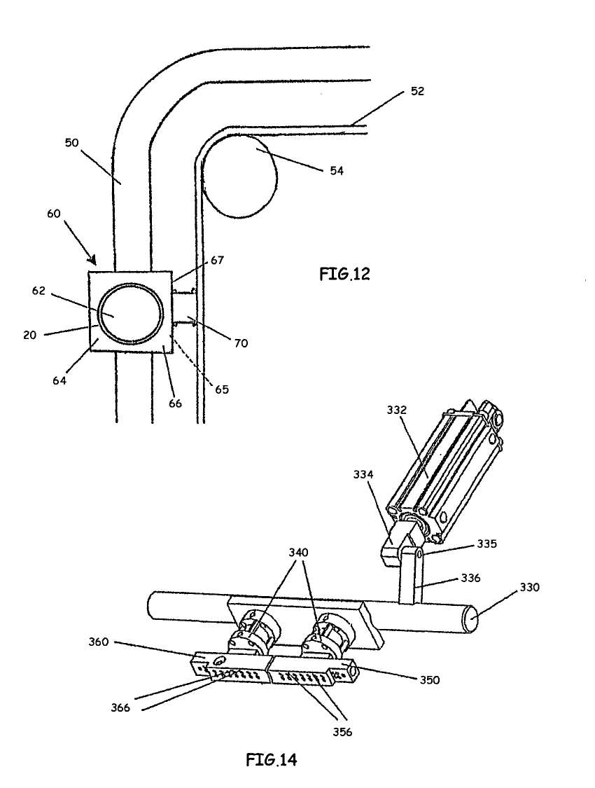

Referring now to FIG. 13, a main drive servo motor 56 is mounted beneath a support frame 18 (see FIG. 6). A main drive motor sprocket 57 is attached to the main drive servo motor 56 such that when the drive motor 56 engages, the motor sprocket 57 rotates. A main drive shaft 59 passes through the support frame 18 at line 19. The drive shaft 59 is oriented in a generally perpendicular direction to the support frame 18, such that part of the drive shaft 59 resides above the support frame 18 and part of the drive shaft 59 resides below the support frame 18. A main drive shaft sprocket 55 is attached to the main drive shaft 59 at a point below the support frame 18. The main drive shaft sprocket 55 is horizontally aligned with the main drive motor sprocket 57. A main drive sprocket 53 is attached to the top of the main drive shaft 59, above the support frame 18. The main drive sprocket 53 is horizontally aligned with the idler sprockets 54, as explained earlier. When the main drive shaft sprocket 55 rotates, both the main drive shaft 59 and the main drive sprocket 53 rotate with it. A main drive motor belt 58 winds around both the main drive motor sprocket 57 and the main drive shaft sprocket 55. When engaged, the main drive servo motor 56 causes the attached main drive motor sprocket 57 to rotate. The rotating main drive motor sprocket 57 turns the main drive motor belt 58, which in turn rotates the main drive shaft sprocket 55. The rotating main drive shaft sprocket 55 is attached to the main drive shaft 59, so when the main drive shaft sprocket 55 rotates, the main drive shaft 59 rotates with it, as does the main drive sprocket 53 attached to the top of the main drive shaft 59. The rotating main drive sprocket 53, in turn, pulls a main drive belt 52 around the main drive sprocket 53 and around the idler sprockets 54. As the main drive belt 52 is pulled around the sprockets 53, 54, the belt 52 itself pulls mandrel assemblies 60 attached to it around a parallel track 50, as explained later. (See FIG. 12.) A PLC unit 120 (See FIGS. 6 and 16) controls the main drive servo motor 56, instructing the motor 56 when to engage and disengage.

Referring to FIG. 12, a plurality of mandrel assemblies 60 are pulled along a track 50. Each mandrel assembly 60 comprises a mandrel 62, a mandrel base 64, and mandrel linkage 70. The bottom side 65 of a mandrel 62 is carried by the track 50, while a top side 66 of the mandrel base 64 supports a mandrel 62. Upon each mandrel 62 is mounted a condom body 20. Mandrel linkage 70 connects a side 67 of the mandrel base 64 that faces a main drive belt 52 with the main drive belt 52. As the main drive belt 52 is drawn around its rectangular path, it pulls the mandrel assemblies 60 along on the parallel track 50. As the mandrel 62 and condom body 20 are pulled around the track 50, they encounter each step of the production cycle in turn.

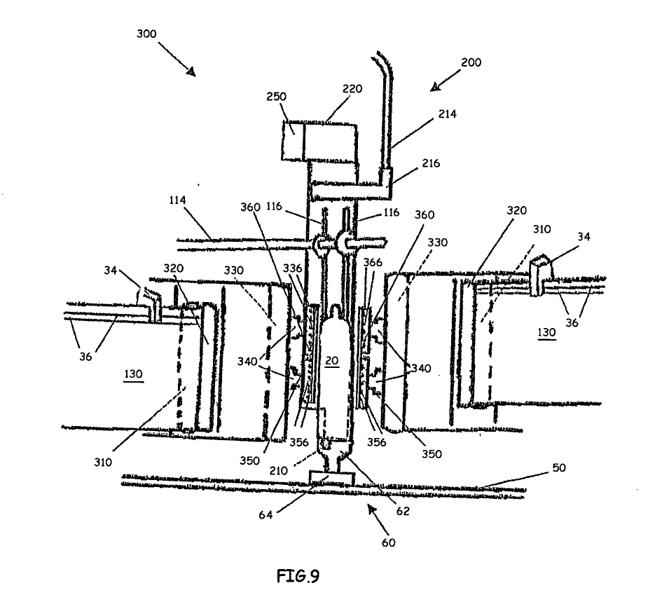

Referring again to FIG. 6 and now also to FIG. 13, the PLC unit 120 instructs a main servo motor 56 to engage. As explained earlier, this action begins the main drive sprocket 53 rotating, which drives the main drive belt 52 around the sprockets 53, 54, which in turn pulls the mandrel assemblies 60 around the track 50. A mandrel assembly 60 and a condom body 20 mounted upon the assembly 60 are pulled along the track 50 and through a conventional condom tester 78. The condom tester 78 tests the condom body 20 for defects. If a defect is found, the condom tester 78 sends information to the PLC unit 120 identifying the specific mandrel assembly 60 upon which the defective condom body 20 is mounted. Ordinarily when a condom body 20 reaches cut and place units 300, each unit 300 applies a plastic strip or tab 32 to an opposing side of the condom body 20. (The specific workings of the cut and place unit 300 are described in more detail later. See FIGS. 9-11.) In the case of a defective condom body 20, however, the PLC unit 120 sends a message to the cut and place units 300 instructing them to ignore the condom body 20. Complying with the instruction, the cut and place units 300 forego applying plastic strips or tabs 32 to the defective condom body 20. A slide and stripper brush assembly 200 then rolls up the defective condom body 20, just as the assembly 200 would do with a non-defective condom body 20. (The specific workings of the slide and stripper assembly 200 are also described in more detail later. See FIGS. 7 and 8.)

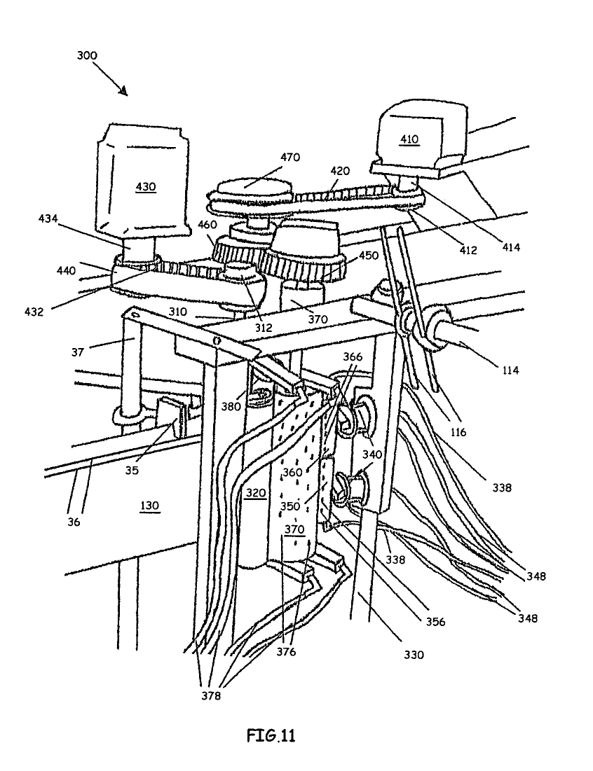

Still referring to FIG. 6, mandrel assemblies 60 upon which condoms are mounted and tested with non-defective condom bodies 20 are continuing to be pulled around the track 50 until each mandrel assembly 60 reaches a slide and stripper assembly 200. At this point, the mandrel assembly 60 and condom body 20 are positioned between two cut and place units 300. Each cut and place unit 300 has previously cut a plastic strip or tab 32 and each now applies that tab 32 to an opposing side of the condom body 20. (See FIGS. 7 and 9-11.) Note that the plastic tab 32 is cut from tab material 130 dispensed from a corresponding metering roll 40. In the present embodiment, the tab material 130 is formed of plastic; however, other types of suitable material 130 have been previously manufactured with narrow bands of texturing 36 on one side of the material 130 toward the edge of the material 130. (See FIGS. 9 and 11.) When the plastic strips 32 are cut from the tab material 130 and integrated with a condom body 20, the textured side 36 of the tabs 32 will all face in one direction. (See FIG. 5.) This allows a user of the condom 10 to determine, through sense of touch, if the tab 32, and thus the condom 10, is oriented in the proper direction. In alternate embodiments, other methods for providing texturing to one side of the tab material 32, toward the edge of the material 130, might be envisioned.

The slide and stripper brush assembly 200 now rolls up the plastic tabs 32 with the condom body 20, leaving the rolled-up condom body 20 balanced on the mandrel assembly 60, as explained later (See FIG. 7.) The PLC unit 120 then instructs each cut and place unit 300 to cut another plastic tab 32 for use by the next condom 20 to be processed. Each unit 300 feeds in tab material 130 then moves into position to be applied to the next condom body 20. (The specific workings of the cut and place unit 300 are described in more detail later. (See FIGS. 9-11.)

Continuing with FIG. 6, the mandrel assembly 60 with the rolled-up condom body 20 and plastic tabs 32 balanced on the end of its mandrel 62 (see FIG. 12) is now pulled about the track 50 until it passes a point across from an air hose 94. The tip of the air hose 94 is pointed at the end of the mandrel 62. As the mandrel 62 and condom body 20 pass, the air hose 94 emits a burst of air to dislodge the rolled-up condom body 20 and its plastic tabs 32 from the end of the mandrel 62. The condom body 20 and its plastic tabs 32 are blown off of the mandrel 62 and into a product collection conduit 90. The condom body 20 and its plastic tabs 32 fall down the conduit 90 and into a product collection bin 96. If the condom body 20 has previously been found to be defective by the condom tester 78, as explained earlier, then the PLC unit 120 sends a message to the air hose 94 to forego emitting a burst of air. The mandrel 62 and rolled-up condom body 20 then continue to be pulled about the track 50 until they pass a point across from a second air hose 84. The tip of the second air hose 84 is also pointed at the end of the mandrel 62. As the mandrel 62 and rolled up condom body 20 pass, the second air hose 84 emits a burst of air to dislodge the rolled-up condom body 20 from the end of the mandrel 62. The condom body 20 is blown off of the mandrel 62 and into a waste collection conduit 80. The condom 20 falls down the conduit 80 and into a waste collection bin 86. A human operator now manually pulls a new condom body 20 onto the mandrel 62. In alternate embodiments, other means for pulling condom bodies 20 onto mandrels 62 are used, such as mechanical means. To aid the operator in pulling condom bodies 20 onto mandrels 62, the support frame 18 upon which the mandrels 62 are mounted is positioned at an angle of approximately twenty-two degrees, as explained earlier. The mandrel 62 and new condom bodies 20 are now pulled around the track 50 through the various stages of the production cycle, as described earlier.

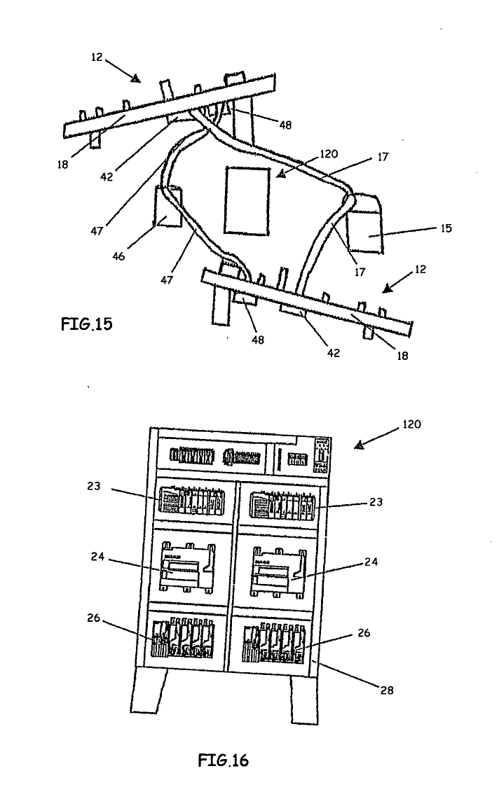

Referring to FIG. 8, a slide and stripper brush assembly 200 comprises a stripper brush 210, a brush motor line 214, a brush motor 216, an air slide 220, an air brush belt 234, a slide rod 240, a linear air cylinder 250, and a slide frame 260. A slide rod 240 is axially aligned along the length of a slide frame 260. The slide rod 240 is attached to the upper surface of the slide frame 260 and generally toward the middle of the slide frame 260. A linear air cylinder 250 is equipped with a hole that runs through the air cylinder 250 and along the length of the air cylinder 250. The slide rod 240 passes through the hole in the linear air cylinder 250 such that the air cylinder 250 rides over the slide rod 240. The top of the linear air cylinder 250 is attached to one end of an air slide 220. When instructed by a PLC unit 120, the linear air cylinder 250 engages, propelling the air slide 220 in one direction along the slide frame 260 until cylinder 250 reaches the opposite end of the slide frame 260. The linear air cylinder 250 attached to the air slide 220 guides the air slide along the slide rod 240. When the linear air cylinder 250 again engages to propel the air slide 220, the air cylinder 250 propels the air slide 220 to the opposite end of the slide frame 260 from where the air slide 220 was residing. An air compressor 46 provides a constant flow of compressed air to a solenoid valve bank 48. (See FIG. 15.) One end of a brush motor line 214 is connected to a brush motor 216. The brush motor 216 is attached to the air slide 220 near the end of the slide 220 that is farthest from a stripper brush 210. The stripper brush 210 is attached to the opposite end of the air slide 220 in the approximate middle of the end of the air slide 220. In alternate embodiments, it is conceivable that more than one stripper brush 210 will be attached to the air slide 220. An air brush belt 234 connects the brush motor 216 with the stripper brush 210. When instructed by the PLC unit 120, a solenoid (not shown) engages to allow air to move through the brush motor line 214 to power the brush motor 216. The brush motor 216 then turns the air brush belt 234 which rotates the stripper brush 210.

Referring now to FIG. 7, when a next mandrel 62 and condom body 20 to be given plastic tabs 32 (see FIG. 9) approach a slide and stripper brush assembly 200, the assembly 200 will be positioned across the path of the oncoming mandrel 62. The assembly 200 is oriented generally parallel to the mandrel 62 and generally perpendicular to the track 50. As the mandrel 62 and condom body 20 are pulled along the track 50 to a point alongside of an air slide 220, and nearly touching it, the PLC unit 120 instructs the main drive servo motor 56 to disengage. This causes the main drive sprocket 53 to stop, which in turn stops the movement of the main drive belt 52, and thereby the movement of the mandrel assemblies 60 along the track 50. (See FIGS. 12 and 13.) This stoppage lasts approximately one second. Note that in some embodiments a stoppage of longer or shorter duration is possible, while in other embodiments is it possible that there will be no stoppage at all. At this point, condom retainer bars 116, and/or cup stop 117 supported by a condom retainer bar rod 114, are positioned at the top end of the mandrel 62, the end farthest from a mandrel base 64. The retainer bars 116 and/or cup stop 117 nearly touch, or barely touch, the top of the condom body 20. Also at this point, a stripper brush 210 is in a position adjacent to the bottom end of the condom body 20, the end closest to the mandrel base 64. The two cut and place units 300 now each place a plastic strip or tab 32 along opposing sides of the condom body 20 and hold them there, as explained in general earlier and as explained in detail later. (See FIG. 6 and FIGS. 9-11.) The PLC unit 120 instructs a solenoid (not shown) to allow air to move through a brush motor line 214 to power a brush motor 216, which in turn sets a stripper brush 210 to rotating. The PLC unit 120 also instructs a linear air cylinder 250 to engage. As the air cylinder 250 begins moving, the attached air slide 220 moves with it, out of the path of the mandrel 62. The air slide 220 moves in a generally straight line away from the track 50 in a direction that is generally perpendicular to the track 50. As explained earlier, the stripper brush 210 is attached to the end of the air slide 220, so that when the air slide 220 moves so does the brush 210. As the air slide 220 begins to move, the rotating brush 210 contacts the base of the condom body 20. The rotational movement of the brush 210 contacts the base of the condom body 20. The rotational movement of the brush 210 against the base of the condom body 20 forces the condom body 20 to begin rolling up. As the condom body 20 does so, the plastic tabs 32 become rolled up inside the condom side wall 14. The rotating brush 210 continues toward the top of the condom body 20, rolling up the condom body 20 and plastic tabs 32 as it climbs. Initially, the plastic tabs 32 are held next to the condom side walls 14 of the condom body 20 by the cut and place units 300, as mentioned earlier. The cut and place units 300 gradually release the plastic strips or tabs 32, however, as the rotating brush 210 rolls up the tabs 32 with the condom body 20. (The specific workings of the cut and place units 300 are described in more detail later. See FIGS. 9-11.) The air slide 220 continues moving in a generally perpendicular direction to the track 50, and away from the track 50 until the air slide 220 and attached stripper brush 210 have moved out of the path of the mandrel 62. At this point, the stripper brush 210 comes away from the top of the mandrel 62, leaving behind the rolled-up condom 110 and plastic tabs 120. The PLC unit 120 then instructs the solenoid (not shown) to shut off the air supply to the brush motor line 214, thereby disengaging the brush motor 216 so that the brush 210 ceases to rotate. The rolled-up condom 20 and tabs 32, propelled to the end of the mandrel 62 by the rotational motion of the brush 210, contact the condom retainer bars 116 and/or cup stop 117 and stop. In this way, the retainer bars 116 and/or cup stop 117 prevent the rolled-up condom 20 and tabs 32 from stripping off the end of the mandrel 62. With the slide and stripper brush assembly 200 now withdrawn out of the path of the mandrel assemblies 62, the PLC unit 120 instructs the main servo motor 56 to reengage. This restarts the main drive sprocket 53 rotating, which propels the main drive belt 52, which in turn pulls the mandrel assemblies 60 along the track 50. (See FIGS. 12 and 13.) As mentioned earlier, the time that the main drive servo motor 56 has been disengaged totals approximately one second. Once the current mandrel 62 has cleared the path of the slide and stripper brush assembly 200, the PLC unit 120 instructs the linear air cylinder 250 to engage, thereby moving the slide and stripper brush assembly 200 back to it's former position, blocking the path of the next oncoming mandrel 62.

Referring now to FIG. 9, the function of a cut and place unit 300 is to cut a plastic tab 32 from tab material 130 and place the plastic tab 32 along side of a condom body 20. Since a plastic tab 32 is required for each side of a condom body 20, another cut and place unit 300 is needed to apply a second tab 32 to the opposing side of the condom 110. The two cut and place units 300 function in substantially the same manner, except that one unit 300 operates from one side of the condom body 20 while the other unit operates from the opposing side of the condom body 20.

Referring to FIG. 11, a cut and place unit 300 comprises a material guide 35, a guide support arm 37, a feed roll 310, a nip roll 320, a tab rotation bar 330, transfer bar lines 338, transfer bar air cylinders 340, transfer bar air cylinder lines 348, a lower tab transfer bar 350, an upper tab transfer bar 360, a vacuum anvil roll 370, vacuum anvil roll lines 378, a knife roll 380, a cut and place drive servo motor 410, a cut and place drive belt 420, a feed roll servo motor 430, a feed roll drive belt 440, a vacuum anvil roll gear 450, a knife roll gear 460, and a knife roll drive sprocket 470. Two transfer bar air cylinders 340 are attached to a tab rotation bar 330. The cylinders 340 reside near one another and are axially aligned along the length of the bar 330. One end of the first cylinder 340 is attached to the rotation bar 330, while the opposite end is attached to an upper tab transfer bar 360. Similarly, one end of the second air cylinder 340 is attached to the rotation bar 330, while the opposite end is attached to a lower tab transfer bar 350. An air compressor 46 provides a constant flow of compressed air to a solenoid valve bank 48. (See FIG. 15.) A first pair of transfer bar air cylinder lines 348 connect a first transfer bar air cylinder 340 to the solenoid valve bank 48. Likewise, a second pair of transfer bar air cylinder lines 348 connect a second transfer bar air cylinder 340 to the solenoid valve bank 48. When instructed by the PLC unit 120 (see FIGS. 6 and 16), a solenoid (not shown) engages to allow air to move through a given pair of lines 348. Depending upon the nature of the instruction given by the PLC unit 120, the movement of air through the pair of lines 348 connected to an air cylinder 340 either causes the air cylinder 340 to move its attached transfer bar 350, 360 away from the tab rotation bar 330 in a generally perpendicular direction, or causes the air cylinder 340 to move its attached transfer bar 350, 360 back toward the tab rotation bar 330 along the reverse path. A blower 15 provides continual suction to a vacuum manifold 42. (See FIG. 15.) Four vacuum anvil roll lines 378 connect a vacuum anvil roll 370 to the vacuum manifold 42. Continual suction supplied by the vacuum manifold 42 causes air to be drawn through the lines 378 and, thus, through perforations 376 in the surface 373 (see FIG. 9) of the vacuum anvil roll 370, thereby providing the anvil roll 370 with continual suction. This suction holds a plastic tab 32 flat against the surface 373 of the anvil roll 370. (Note that the anvil roll 370 is configured into four quadrants, with each quadrant measuring approximately ninety degrees in area. Suction is provided to only one quadrant of the roll 370 at any one time.) A first transfer bar line 338 connects a lower tab transfer bar 350 to the vacuum manifold 42. Likewise, a second transfer bar line 338 connects an upper tab transfer bar 360 to the vacuum manifold 42. When instructed by the PLC unit 120, a solenoid (not shown) engages to allow air to move through a given line 338. Continual suction supplied by the vacuum manifold 42 causes air to be drawn through line 338 and, thus, through perforations 356,366 in the surface of the respective transfer bar 350, 360, thereby providing suction to the respective transfer bar 350, 360.

Referring to FIG. 14, one end of a pivot bar 336 is attached to an end of a pivot air cylinder arm 334 by a fastener 335. The fastener 335 attaches to the pivot air cylinder arm 334 and to the pivot bar 336 in such a way as to allow the pivot bar 336 to pivot about the fastener 335. The other end of the pivot air cylinder arm 334 that is opposite the end of the arm 334 attached to the pivot bar 336 is telescopically attached to a pivot air cylinder 332. Being as the pivot air cylinder arm 334 is telescopically attached to the pivot air cylinder 332, the pivot air cylinder are 334 can be drawn inside the pivot air cylinder 332 or extended outwardly from the pivot air cylinder 332. When instructed by a PLC unit 120 (see FIGS. 6 and 16), the pivot air cylinder 332 moves the pivot air cylinder arm 334 either inwardly or outwardly, as instructed. The inward or outward motion of the pivot air cylinder arm 334 causes the pivot bar 336 to pivot about the fastener 335. The pivoting motion of the pivot bar 336 around the faster 335 causes the tab rotation bar 330 to rotate a given number of degrees, depending upon the distance the pivot air cylinder arm 334 travels.

Referring again to FIG. 9, after a slide and stripper brush assembly 220 has rolled up a condom 110 and plastic tabs 120 together, as explained earlier, the PLC unit 120 instructs solenoids (not shown) to allow air to move through both transfer bar lines 338, thereby providing suction to the transfer bars 350, 360, as explained earlier. The PLC unit 120 also instructs a pivot air cylinder 332 to rotate a tab rotation bar 330. (See FIG. 14 for details.) The rotating tab rotation bar 330 rotates the lower and upper tab transfer bars 350, 360 approximately ninety degrees, positioning the perforated surfaces 356, 366 of the transfer bars 350, 360 parallel to, and adjacent, a perforated surface 373 (see FIGS. 10 and 11) of a vacuum anvil roll 370. The suction exerted by the transfer bars 350, 360 is greater than that exerted by the vacuum anvil roll 370. As a result, a plastic tab 32 held flat on the surface 373 of the anvil roll 370 is sucked away from the roll 370 and onto the surfaces of the transfer bars 350, 360. The plastic tab 32 is now held flat atop the transfer bars 350, 360 with the plastic tab 32 extending across substantially the entire length of the perforated surfaces 356, 366 of both of the transfer bars 350, 360. The PLC unit 120 instructs the pivot air cylinder 332 to rotate the tab rotation bar 330 back. The rotating tab rotation bar 330 rotates the upper and lower tab transfer bars 350, 360 are now in position to place the plastic tab 32 on the next condom body 20 to arrive. Note that FIG. 9 depicts the apparatus 12 in this position, i.e. the transfer bars 350, 360 back in their starting positions ready to place the plastic tab 32 on a newly-arrived condom body 20.

Recalling now an earlier explanation, a mandrel 62 and condom body 20 are pulled along the track 50 until they reach a point alongside an air slide 220, nearly touching the air slide 220. At this point, the PLC unit 120 instructs a main drive servo motor 56 to disengage. This causes the main drive sprocket 53 to stop, which in turn stops the movement of the main drive belt 52, and thereby the movement of the mandrel assemblies 60 along the track 50. (See FIGS. 12 and 13.) The condom body 20 is now in a position to accept the plastic tab 32 that the transfer bars 350, 360 are holding.

Returning to FIG. 9, the PLC unit 120 instructs solenoids (not shown) to allow air to move through two pairs of transfer bar air cylinder lines 348 (see FIG. 11), causing two air cylinders 340 to extend outwardly the transfer bars 350, 360 to the side of a condom body 20. The transfer bars 350, 360 press a plastic tab 32 alongside of the condom body 20 and hold the tab 32 there. The plastic tab 32 extends from the top of the condom body 20, the top being defined as that point farthest from the mandrel base 64, to a point somewhat near the bottom of the condom body 20, the bottom being defined as that point closest to the mandrel base 64. Note that a material guide 35 can be manually repositioned along a guide support bar 37 to alter the positioning of plastic tabs 120 along the condom side walls 14, as explained later. A slide and stripper brush assembly 200 now begins to roll up the condom body 20 and plastic tabs 32 together, as explained earlier. Just before the rolled-up condom body 20 reaches the lower tab transfer bar 350, the PLC unit 120 instructs a solenoid (not shown) to allow air to move through the associated pair of transfer bar air cylinder lines 348, causing the air cylinder 340 to retract the transfer bar 350 from along the side of the condom body 20. As this is done, the PLC unit 120 further instructs the associated solenoid to cut suction to the lower tab transfer bar 350 and to direct a blast of air through the perforations 356 of the bar 350 instead. This blast of air pushes down on the plastic tab 32, holding the tab 32 against the condom body 20 as it is rolled up over this portion of the plastic tab 32. Similarly, just before the rolled-up condom body 20 reaches the upper tab transfer bar 360, the PLC unit 120 instructs a solenoid (not shown) to allow air to move through the associated pair of transfer bar air cylinder lines 348, causing the air cylinder 340 to retract the transfer bar 360 from the side of the condom body 20. As this is done, the PLC unit 120 further instructs the associated solenoid to cut suction to the upper tab transfer bar 360. This procedure is performed by two cut and place units 300. One cut and place unit 300 is positioned on one side of the condom body 20, while the other cut and place unit 300 side positioned on the opposing side of the condom body 20, as mentioned earlier. In this way, a plastic tab 32 is applied to opposing sides of the condom body 20 at the same time. As explained earlier, the slide and stripper brush assembly 200 now finishes rolling up the plastic tabs 32 with the condom body 20. (See FIG. 7.)

With the transfer bars 350, 360 having taken the plastic tab 32 from the anvil roll 370, then anvil roll 370 must now be supplied with a new plastic tab 32. The anvil roll 370 must also be moved into a position that will make the new plastic tab 120 available to the transfer bars 350, 360 when they next need a tab 32, as explained earlier. The process of cutting a new plastic tab 32 and making it available to the transfer bars 350, 360 is explained next.

Referring now to FIG. 11, a cut and place drive shaft 414 is attached to a cut and place drive servo motor 410 such that when the drive motor 410 engages, the drive shaft 414 begins to turn. Attached to the end of the drive shaft 414 is a cut and place drive sprocket 412. The drive sprocket 412 turns as the drive shaft 414 turns. A cut and place drive belt 420 winds around the cut and place drive sprocket 412 and also around a knife roll drive sprocket 470. When the drive belt 420 turns, so do the cut and place drive sprocket 412 and the knife roll drive sprocket 470. The knife roll drive sprocket 470 is attached to the end of a knife roll shaft 384 (see FIG. 10), which is part of a knife roll 380. The knife roll shaft 384 turns as the knife roll drive sprocket 470 turns. A knife roll gear 460 is also attached to the knife roll shaft 384 at a point below the knife roll drive sprocket 470. When the sprocket 470 turns, both the knife roll gear 460 and the knife roll 380 turn with it. The knife roll gear 460 is also meshed with a vacuum anvil roll gear 450. The vacuum anvil roll gear 450 is attached to a vacuum anvil roll shaft 374 (see FIG. 10), which is part of a vacuum anvil roll 370. When the knife roll gear 460 turns, it turns the vacuum anvil roll gear 450. Since the vacuum anvil roll gear 450 is attached to the vacuum anvil roll shaft 374, when the gear 450 turns so does the vacuum anvil roll 370.

Still referring to FIG. 11, the process of cutting a new plastic tab 32 and making it available to transfer bars 350, 360 begins at the moment when a tab rotation bar 330 begins rotating the transfer bars 350, 360 away from a position adjacent to a vacuum anvil roll 370 and back to the original starting positions of the transfer bars 350, 360, as explained earlier. (See FIG. 9.) As that movement is occurring, the PLC unit 120 instructs a cut and place drive servo motor 410 to engage. When the drive motor 410 engages, an attached cut and place drive shaft 414 begins turning. A cut and place drive sprocket 412 attached to the drive shaft 414 also begins turning with the drive shaft 414. The rotation of the cut and place drive sprocket 412 pulls a cut and place drive belt 420 around the drive sprocket 412. As the drive belt 420 is being pulled around the rotating drive sprocket 412, it is also being pulled around the knife roll drive sprocket 470 causes the sprocket 470 to rotate. As the knife roll sprocket 470 rotates, both a knife roll gear 460 and a knife roll 380 rotate with it. Since the knife roll gear 460 is also meshed with a vacuum anvil roll gear 450, the anvil roll gear 450 rotates as the knife roll gear 460 rotates. Also, since the rotating anvil roll gear 450 is attached to a vacuum anvil roll 370, the rotating anvil roll gear 450 causes the anvil roll 370 to rotate as the gear 450 rotates. In this way, when the PLC unit 120 instructs the cut and place drive servo motor 410 to engage, the knife roll 380 and vacuum anvil roll 370 are made to rotate together in tandem.

Continuing to refer to FIG. 11, a feed roll motor shaft 434 is attached to a feed roll servo motor 430 such that when the motor 430 engages, the feed roll motor shaft 434 begins to turn. Attached to the end of the feed roll motor shaft 434 is a feed roll motor sprocket 432. The feed motor sprocket 432 turns as the motor shaft 434 turns. A feed roll drive belt 440 winds around the feed roll motor sprocket 432 and also around a feed roll sprocket 312. When the drive belt 440 turns, so do the two sprockets 432, 312. The feed roll sprocket 312 is attached to the end of a feed roll 310. The feed roll 310 turns as the feed roll sprocket 312 turns.

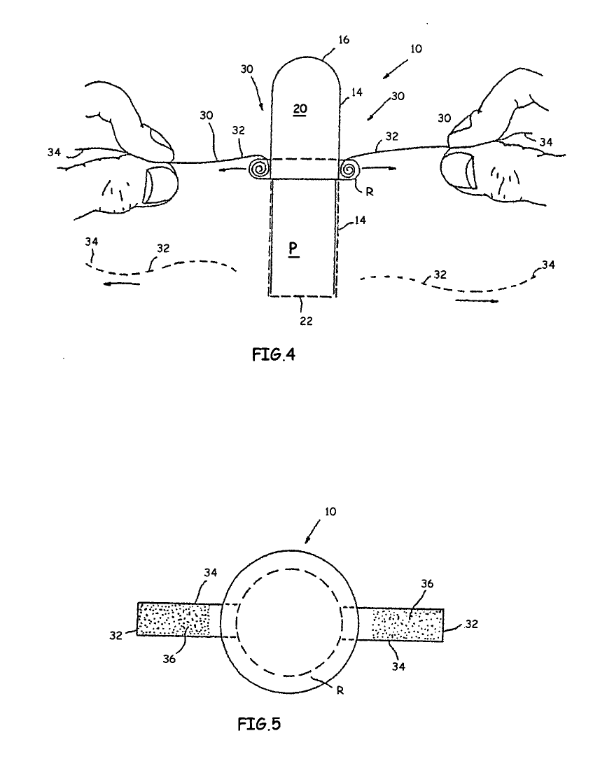

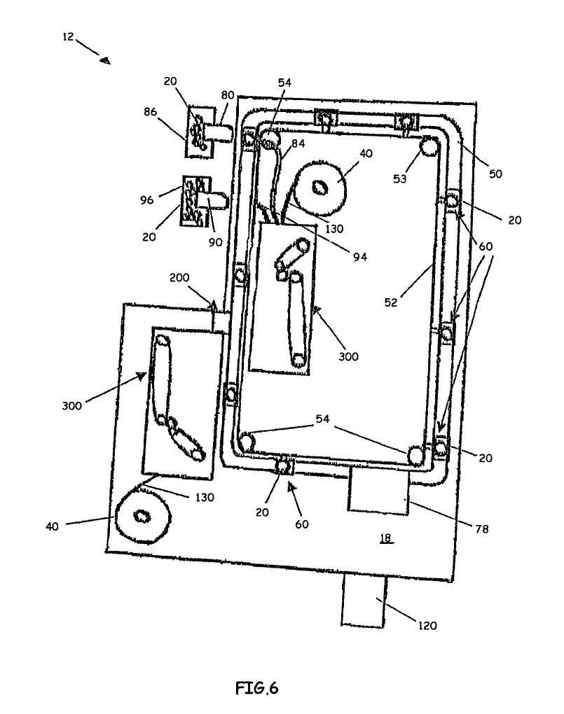

Further referring to FIG. 11, at approximately the same moment the PLC unit 120 instructs a cut and place drive servo motor 410 to engage, which leads to a vacuum anvil roll 370 and a knife roll 380 rotating together in tandem, as explained earlier, the PLC unit 120 also instructs a feed roll servo motor 430 to engage. When the feed roll servo motor 430 engages, an attached feed roll motor shaft 434 begins turning. A feed roll sprocket 312 attached to the feed roll motor shaft 434 begins turning. A feed roll sprocket 312 attached to the feed roll motor shaft 434 also begins turning with the shaft 434. The rotation of the feed roll motor sprocket 432 pulls a feed roll drive belt 440 around the sprocket 432. As the feed roll drive belt 440 is being pulled around the rotating feed roll motor sprocket 434, it is also being pulled around a feed roll sprocket 312. The action of the feed roll drive belt 440 being pulled around the feed roll sprocket 312 causes the sprocket 312 to rotate. As the feed roll sprocket 312 rotates, the feed roll 310 rotates with it. In this way, when the PLC unit 120 instructs the feed roll servo motor 430 to engage, the feed roll 310 is made to rotate. The rotating feed roll 310 pulls tab material 130 through a material guide 35, advancing the material 130 further onto a vacuum anvil roll 370. Note that only an amount of tab material 130 equivalent to the width of a plastic tab 32 (see FIGS. 4 and 5) is advanced onto the anvil roll 370. The result of this is that the feed roll 310 need rotate only slightly amount of tab material 130. The material guide 34 is attached to a guide support bar 38. The guide 34 can be manually repositioned along the guide support 38 to alter the position of plastic tabs 32 along the sides of condom bodies 20. When the guide 34 is repositioned on the support bar 38 to a location closer to the top condom body 20, plastic tabs 32 will be positioned nearer the top of condom bodies 20. When the guide 34 is repositioned on the support bar 38 to a location closer to the bottom of a condom body 20, plastic tabs 32 will be positioned nearer the bottom of condoms 110. Mounted adjacent to the feed roll 310 is a nip roll 320. the two rolls 310, 320 are oriented generally parallel to each other with the tab material 130 lying flat between the rolls 310, 320. The nip roll 320 is equipped with a spring which forces the nip roll 320 against the adjacent feed roll 310. The tab material 130, caught between the two rolls 310, 320, is held snugly in place by the rolls 310, 320. When the tab material 130 is advanced onto the vacuum anvil roll 370, the material 130 is fed between these two rolls 310, 320. In this way, the nip roll 320 and feed roll 310 act together to prevent the tab material 130 from slipping as it is fed into the vacuum anvil roll 370.

Referring now FIG. 10, a vacuum anvil roll 370 comprises an anvil 372, a surface 373, two shafts 374, and a plurality of perforations 376. A barrel 375 forms the central portion of a vacuum anvil roll 370. Two shafts 374 are axially aligned along the length of the barrel 375. A first shaft 374 extends from one of the barrel 375, while a second shaft 374 extends from the opposing end of the barrel 375. In alternate embodiments, there will be only one shaft 374, which will pass through the center of a barrel 375 with each end of the shaft 374 extending beyond each respective end of the barrel 375. In the present embodiment, the two shafts 374 and the barrel 375 are all machined from one piece of material, such as steel. In alternate embodiments, a shaft or shafts 374 are manufactured separately from a barrel 375 and the shaft or shafts 374 then fastened together with the barrel 375 to form one functional unit. An anvil 372, narrow in width, in inset into a surface 373 of the barrel 375. The top surface of the anvil 372 is generally flush with the surface 373 of the barrel 375. The top surface of the anvil 372 can also be inset below the surface 373 of the barrel 375 or raised somewhat above the surface 373. This variation in depth is possible due to the fact that the fact the position of a knife 382 on the surface 383 of a knife roll 380 can be adjacent to compensate for the depth of the anvil 372. (The interaction of the anvil 372 and the knife 382 is explained later.) The anvil 372 extends along the entire length of the barrel 375. The anvil 372 provides the platform upon which tab material 130 will be cut to create a plastic tab 32. The anvil 372 is constructed of a material hard enough to withstand repeated encounters with the cutting edge of knife 382. An example of a suitable material would be hardened carbide steel. It is to be understood, however, that the anvil 372 will need to be replaced from time to time due to excessive wear. Perforations 376 through which air can be drawn to provide suction appear in both the surface 373 of the barrel 375 and in the anvil 372.

Still referring to FIG. 10, a knife roll 380 comprises a knife 382, a surface 383, two shafts 384, and a barrel 385. A barrel 385 forms the central portion of a knife roll 380. Two shafts 384 are axially aligned along the length of the barrel 385. A first shaft 384 extends from one end of the barrel 385, while a second shaft 384 extends from the opposing end of the barrel 385. In alternate embodiments, there will be only one shaft 384, there will be only one shaft 384, which will pass through the center of a barrel 385 with each end of the shaft 384 extending beyond each respective end of the barrel 385. In the present embodiment, the two shafts 384 and the barrel 385 are all machined from one piece of material, such as steel. In alternate embodiments, a shaft or shafts 384 are manufactured separately from a barrel 385 and the shaft 384 then fastened together with the barrel 385 to form one functional unit. A knife 382 is attached to the surface 383 of the barrel 385 and extends along the entire length of the barrel 385. The knife 382 will be used to create plastic tabs 32 by cutting tab material 130 that is held in place on the surface 373 of the vacuum anvil roll 370, as explained later.

Recalling now an earlier explanation, when the vacuum anvil roll 370 and the knife roll 380 begin rotating, a feed roll 310 also begins rotating. The rotating feed roll 310 advances a small amount of tab material 130 on the anvil roll 370, even as the anvil roll 370 itself rotates. The amount of tab material 130 advance onto the anvil roll 370 is equivalent to the width of a plastic tab 32 to be cut. Since only a small amount of tab material 130 needs to be advanced on the anvil roll 370, the feed roll 310 need rotate only slightly before stopping. Meanwhile, the spring-loaded nip roll 320 presses the tab material 130 against the feed roll 310, keeping the tab material 130 from slipping. The rotating action of the anvil roll 370, the continual suction applied to the surface 373 of the anvil roll 370, and the action of the feed roll 310 and nip roll 320 acting together to hold the tab material 130 snugly between them, all work to pull the tab material 130 taut across the surface 373 of the anvil roll 370 as the anvil roll 370 rotates.

Continuing now with FIG. 10, once a feed roll 310 has advanced onto a vacuum anvil roll 370 an amount of tab material 130 equivalent to the width of a plastic tab 32, the PLC unit 120 instructs a feed roll servo motor 430 (see FIG. 11) to disengage. This action stops the feed roll 310 from rotating which, in turn, halts the advancement of tab material 130. The vacuum anvil roll 370 and a knife roll 380 are aligned such that as the rolls 370, 380 rotate in tandem, a knife 382 on the knife roll 380 contacts an anvil roll 370 during their synchronized rotation. Note that the angle of the knife 382 to the anvil 372 can be adjusted manually during the setup procedure for the apparatus 12. Tab material 130 lies drawn over the anvil 372 of the anvil roll 370 as the knife 382 arrives. The knife 382 cuts through the tab material 130 at the point where the material 130 crosses the anvil 372. The resulting cut leaves behind on the surfaces 373 of the anvil roll 370 a newly-created plastic tab 32. The plastic tab 32 is held flat against the surface 373 of the anvil roll 370 by suction. The vacuum anvil roll 370 and the knife roll 380 continue to rotate until the two rolls 370, 380 have completed one full rotation. The tab material 130 remains held in place by the feed roll 310 and a nip roll 320 as the anvil roll 370 rotates. At this point, the PLC unit 120 instructs a cut and place drive servo motor 410 (see FIG. 11) to disengage. This stops the rotation of the vacuum anvil roll 370 and the knife roll 380. The vacuum anvil roll 370 is now in a position to transfer the newly-created plastic tab 32 to transfer bars 350, 360 (see FIG. 9) when next the transfer bars 350, 360 are in a position to accept the plastic tab 32.

FIG. 15 shows another embodiment of a method and apparatus for manufacturing tab applicator assemblies 30 and properly integrating them with existing condom bodies 20.

Referring to FIG. 15, the present embodiment comprises two apparatus 12, a blower 15, two vacuum ducts 17, two support frames 18, a programmable logic controller (“PLC”) unit 120, two vacuum manifolds 42, a means for providing compressed air 46, and two solenoid valve banks 48. Each apparatus 12 is mounted upon a support frame 18. A PLC unit 120 is positioned between the two apparatus 12 and frames 18. The PLC unit 120 is connected by standard means to various components and mechanisms of each apparatus 12 so as to control and coordinate the overall operation of each apparatus 12. (See FIG. 16.) In the alternative embodiments, a PLC unit 120 provides control and coordination for more than two apparati 12. In other embodiments, a PLC unit 120 provides control and coordination for just one apparatus 12. A blower 15 also resides between the two apparatus 12. One end of a first vacuum duct 17 is connected to the blower 15 while the other end of the vacuum duct 17 is attached to a first vacuum manifold 42. The first vacuum duct 17 connects to the first vacuum manifold 42 on the underside of a first support frame 18. One end of a second vacuum duct 17 is also connected to the blower 15 while the other end of the second vacuum duct 17 is attached to a second vacuum manifold 42. The second vacuum duct 17 connects to the second vacuum manifold 42 on the underside of a second support frame 18. Vacuum lines from various components and mechanisms of each apparatus 10 connect to each respective vacuum manifold 42, as explained later. Each respective manifold 42 provides these components and mechanisms with connection points to the vacuum duct 17. The blower 15 delivers continual suction to each vacuum duct 17. Each vacuum duct 17, through its respective vacuum manifold 42, delivers this suction to the vacuum lines connected to the manifold 42. In alternate embodiments, a blower 15 and vacuum ducts 17 provide suction to more than two apparati 12. In other embodiments, a blower 15 employs one or more vacuum ducts 17 to provide suction to just one apparatus 12.

Continuing with FIG. 15, a means for providing compressed air 46, such as an air compressor, is available to each apparatus 12. The air compressor 46 is envisioned by the present embodiment as a stand-alone unit, independent of an apparatus 12. An air compressor 46 most generally supplies compressed air by way of air hoses 47. One end of a first air hose 47 is connected to the air compressor to the air compressor 46 while the other end of the air compressor 46 while the other end of the first air hose 47 is attached to a first solenoid valve bank 48. The first air hose 47 connects to the first solenoid valve bank 48 on the underside of a first support frame 18. One end of a second air hose 47 is also connected to the air compressor 46 while the other end of the second air hose 47 is attached to a second solenoid valve bank 48. The second air hose 47 connects to the second solenoid valve bank 48 on the underside of a second support frame 18. Air lines from various components and mechanisms of each apparatus 12 connected to each respective solenoid valve bank 48, as explained later. Each respective valve bank 48 provides these components and mechanisms with connection points to the air hose 47. The air compressor 46 delivers a continual flow of compressed air to each air hose 47. Each air hose 47, through its respective solenoid valve bank 48, delivers this flow of air to the air lines connected to valve bank 48. In the present embodiment, an air compressor 46 services two apparati 12. In alternate embodiments, an air compressor 46 services one apparatus 12. In other embodiments, an air compressor 46 can service multiple apparati 12.

Referring to FIG. 16, a PLC unit 120 can be used to control and coordinate the operation of two apparati 12, but in practice it is preferable for maintenance purposes to use a separate PLC unit 120 for each apparatus. As mentioned earlier, in alternate embodiments a PLC unit 120 is configured to operate more than two apparati 12. In other embodiments, a PLC unit 120 is configured to operate more than two apparati 12. The PLC unit 120 comprises two PLC controllers with input, output, and communication modules 23; two line interface modules 24; two banks of servo axis modules 26; and a cabinet 28. The cabinet 28 contains the equipment necessary to run two apparati 10. The cabinet 28 contains two PLC controllers with input, output, and communication modules 23; two line interface modules 24; and two banks of servo axis modules 26. One PLC controller 120, one line interface module 24, and one bank servo axis modules 26 are used to run each apparatus 12. The PLC controller 21 is a computer that controls and coordinates the operation of an apparatus 12 by issuing instructions to the servo axis modules 26. Commands from PLC controller 21 instruct the bank of servo axis modules 26 to supply power to various servo motors that run various components and mechanisms of the apparatus 12. The line interface module 24 is a device that takes primary supply voltage from the system, converts the voltage to the correct format for use by the servo axis modules 26, and feeds the power through an emergency stop contractor to the servo axis modules 26. The line interface module 24 also communicates emergency stop information to the PLC controller 21 in the event of a problem. Through its components, the PLC unit 120 controls and coordinates the operation of two apparati 12.

The next aspect of the present invention is the packaging apparatus or machine 500 as seen schematically in FIG. 17. The packaging machine 500 encloses the rolled condom and integrated condom applicator assembly in a wrapper as a sealed package. The packaging machine 500 is also referred to as a “foiler” because it packages the combined condom and applicator assembly (the “product assembly”) in a foil type material used for the wrapper. The wrapper can also be made from polyester film. In any event the wrapper material is fed into the packaging machine in the form of two continuous rolls 502 and 504, each having the same width that is slightly wider than the width of the product assembly. The webs of the two rolls 502, 504 are preprinted with the packaging design, trademark of the product, directions for use, warnings, etc. One roll 502 feeds a web 506 that becomes the front side of the package, while the second roll 504 feeds a web 508 that becomes the back side of the package. Thus the printing on the front side is different than the printing on the second side, and each has a repeat length that is defined by the package size.

The foil feed assembly 510 features two floating rollers 512 and 514 that move up and down to absorb the start and stop of the webs 506 and 508 which is caused downstream by processing one package at a time as later explained. After passing the floating roller 512, web 506 which becomes the top of the package passes an edge printer 516 that prints on the edge of web 506 a product lot number and other identifying data. The two webs 506 and 508 are first joined together with a presealer 518 which uses heat and pressure to form a first edge of the package. The webs 506 and 508 must be stopped to perform this preseal. Every step after this point is performed one package at a time, which is the reason for the two floating rollers 512 and 514 described above.

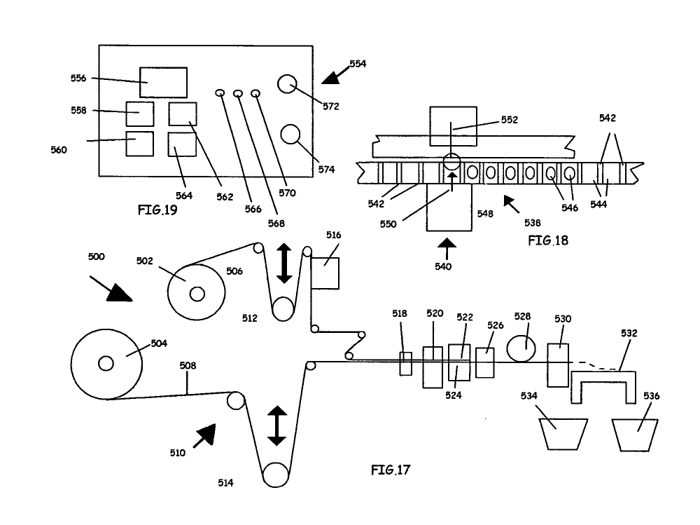

Product assemblies are then inserted between webs 506 and 508 next to the first edge of the package at the first station 520 of the package forming site as more fully described in connection with FIG. 18. The second station 522 of the package forming site is a four side sealer 524 that confirms the one side preseal and supplies three more side seals using heat and pressure to complete closing of the package with the product assembly inside. This is followed by a cooling position 526 to prevent the residual heat in the package from the four side sealer 524 from damaging the condom inside of the package. The next position is a guide roller 528 to accurately locate the package web for entry into the perforator/cutter 530. The perforator/cutter 530 has the dual role of placing a perforation between adjoining packages so they can be readily separated by the consumer, and also cutting packages apart from each other. Thus for a product web intended for placement in a three pack box, every third perforation is cut through by the cutter function of the perforator/cutter 530. The packages then preferably drop to an inspection table 532 where an inspector checks the integrity of the package, the lot number printing, and deposits the packages in an approved bin 534 or reject bin 536. The packages are then boxed and placed in cartons with user instructions for shipping (not shown). The marketing aspect of the invention is structured using manufacturer's representatives hired by the owner of this invention and the foregoing equipment.

FIG. 18 is a top view partial schematic showing the condom sectioned feeder belt 538 and pusher/eccentric tong position 540. The feeder belt 538 is sectioned by a multiplicity of transverse separator members 542. Each section 544 is designed to hold one product assembly 546 separate from other product assemblies 546 and in a precise location. A packaging machine operator places each product assembly 546 in its own section 544 as the feeder belt 538 moves one section 544 at a time. When a given product assembly 548 reaches the pusher/eccentric tong position 540, it is moved laterally from the feeder belt 538 to between the two webs 506 and 508. The pusher/eccentric tong position 540 is located at the first station 520 of the package forming site described in regard to FIG. 17. The pusher 550 literally pushes the product assembly 548 out of section 544, while the eccentric tong 552 is moved in a flattened configuration to the product assembly 548, rotates about its own axis to expose a tong, and the tong hooks the product assembly 548 and pulls the product assembly 548 from the feeder belt 538 to between the two webs 506 and 508 in cooperation with the pusher 550.

Turning finally to FIG. 19, a front view schematic of the foiler packaging machine 500 control panel 554 is seen. The top left instrument is a data display 556 showing batch and total product count. The lower left cluster of screens showing upper and lower preheater temperatures, 558 and 560, and upper and lower main sealing temperatures, 562 and 564. To the right, is a three button grouping for start, reset, and stop functions, 566, 568, and 570. At the far right is a rotational on and off power switch 572. Beneath power switch 572 is emergency stop button 574.

### Instructions Regarding the Method of Use of the Condom with Integrated Applicator

The inventive process finally includes instructing the consuming public how to use condom 10. The instructions provide, first, that the user grasps each tab end segment 34 between the thumb and the index finger of the closest hand and touches the tactile textured surfaces 36 to orient the condom in the proper direction because if it is not properly oriented, it will unroll in the wrong direction. If necessary, the condom is turned over. See FIG. 5. The user then places condom end wall 16 against the distal end of the penis P in the conventional way, so that tubular side wall roll R is directed away from the user. Then the user grasps each tab end segment 34 between the thumb and the index finger of the closest hand and pulls tab end segments 34 radially outward from the sides of the penis P. See FIG. 4. This single action causes the rolled side wall 14 to quickly unroll and advance longitudinally over the penis P. Once tubular side wall 14 is fully unrolled, strips or tabs 32 are necessarily unrolled as well and freed from condom body 20, so that strips or tabs 32 may be lifted away from condom body 20 by the user. Strips or tabs 32 are later collected and discarded together with the used condom body 20.

While the invention has been described, disclosed, illustrated and shown in various terms or certain embodiments or modifications which it has assumed in practice, the scope of the invention is not intended to be, nor should it be deemed to be, limited thereby and such other modifications or embodiments as may be suggested by the teachings herein are particularly reserved especially as they fall within the breadth and scope of the claims here appended.

1-18. (canceled)

19. A process for the manufacture, testing, assembly, and packaging of a condom with an integrated applicator comprising:
manufacturing of a condom of a type that can be rolled for packaging purposes;
manufacturing of at least one flexible strip to be used as an applicator tab;
assembling the condom and the at least one applicator tab together by mechanically rolling the condom and the at least one applicator tab together to form a rolled condom and integrated condom applicator assembly (product assembly); and
packaging the product assembly by mechanically sealing it in a wrapper.

20. The process of claim 19 in which the product assembly is comprising a condom rolled up with two applicator tabs positioned diametrically opposite each other.

21. The process of claim 20 in which the applicator tabs are further comprising gripping end tab segments that protrude from product assembly and have touch distinctive tactile surfaces on one side of the gripping end tab segments.

22. The process of claim 21 in which touch distinctive tactile surfaces facilitate using a user's sense of touch for orienting in the dark the condom in a proper direction for unrolling the condom on a penis.

23. The process of claim 20 in which mechanically rolling the condom and the applicator tabs together to form product assemblies further comprises:
applying the condoms to mandrels in a machine spaced apart on and moved about a track by a main drive belt;
testing the integrity of each condom for defects;
rejecting and discarding defective condoms;
cutting flexible strips to be used as applicator tabs;
placing the applicator tabs adjacent condoms on mandrels;
rolling up condoms with applicator tabs integrated therewith; and
removing the rolled up product assemblies from the mandrels.

24. The process of claim 20 in which packaging the product assembly by mechanically sealing it in a wrapper further comprises:
unrolling preprinted upper and lower rolls to form a continuous upper web and a continuous lower web to later provide respectively a package top and a package bottom;
inserting the product assembly between the upper web and the lower web;
sealing the upper web to the lower web on four sides around the product assembly; and
perforating side seals between adjoining product assemblies to facilitate separation of packages from each other.

25. The process of claim 24 which further comprises presealing a first edge of the package before inserting the product assembly between the upper web and lower web.

26. The process of claim 24 which further comprises cooling the package after the four side sealer.

27. The process of claim 24 which further comprises cutting groups of packages from each other such as to facilitate filling three pack boxes.

28. A process for the manufacture, testing, assembly, packaging, sale and method of use of condoms with integrated applicators comprising:
manufacturing of condoms of a type that can be rolled for packaging purposes;
manufacturing of two flexible strips per condom to be used as applicator tabs;
assembling the condoms and the two applicator tabs together by mechanically rolling the condoms and the applicator tabs together to form rolled condom and integrated condom applicator assemblies (product assemblies);
packaging the product assemblies by mechanically sealing them in wrappers to form product packages (packs);
boxing the packs, such as in a three pack box with instructions concerning the method of use;
selling the product packages to retailers through manufacturers' representatives.

29. The process of claim 28 in which the applicator tabs are further comprising gripping end tab segments that protrude from the product assemblies and have touch distinctive tactile surfaces on one side of the gripping end tab segments.

30. The process of claim 29 in which touch distinctive tactile surfaces facilitate using a user's sense of touch for orienting in the dark the condom in a proper direction for unrolling the condom on a penis.

31. The process of claim 28 in which mechanically rolling the condoms and the applicator tabs together to form product assemblies further comprising:
applying the condoms to mandrels in a machine spaced apart on and moved about a track by a main drive belt;
testing the integrity of each condom for defects;
rejecting and discarding defective condoms;
cutting flexible strips to be used as applicator tabs;
placing the applicator tabs adjacent condoms on mandrels;
rolling up condoms with applicator tabs integrated therewith; and
removing the rolled up product assemblies from the mandrels.

32. The process of claim 28 in which packaging the product assemblies by mechanically sealing them in wrappers further comprises:
unrolling preprinted upper and lower rolls to form a continuous upper web and a continuous lower web to later provide respectively package tops and package bottoms;
inserting product assemblies between the upper web and the lower web;
sealing the upper web to the lower web on four sides around the product assemblies; and
perforating side seals between adjoining product assemblies to facilitate separation of packages from each other.

33. The process of claim 32 which further comprises presealing a first edge of the packages before inserting the product assemblies between the upper web and lower web.

34. The process of claim 32 which further comprises cooling the packages after the four side sealer.

35. The process of claim 32 which further comprises cutting groups of packages from each other such as to facilitate filling three pack boxes.

36. The process of claim 28 in which the instructions for use comprise:
opening the pack and removing the product assembly;
grasping the tab end segments of the applicator tabs that protrude from the rolled condom between the thumb and the index finger of each hand;
touching the tactile textured surfaces on one side of the tab end segments;
orienting the condom in the proper direction because if it is not properly oriented, it will unroll in the wrong direction,
turning over the condom if necessary;
placing the condom end wall against the end of the penis in the conventional way, so that tubular side wall roll is directed away from the user;
pulling the tab end segments radially outward from the sides of the penis causing the rolled side wall to quickly unroll and advance longitudinally over the penis; and
discarding the applicator tabs once tubular side wall of the condom is fully unrolled.
